(19) United States
(12) Patent Application Publication
(10) Pub. No.: US 2025/0228609 A1
(43) Pub. Date: Jul. 17, 2025

(54) ENDOSCOPE TREATMENT TOOL

(71) Applicant: Olympus Corporation, Tokyo (JP)

(72) Inventors: Yasuhito SUGA, Musahino-shi (JP); Yuta MUYARI, Hino-shi (JP); Chika MIYAJIMA, Hachioji-shi (JP); Hiromasa KATO, Tokyo (JP); Takanobu YANAI, Hachioji-shi (JP)

(21) Appl. No.: 19/015,608

(22) Filed: Jan. 9, 2025

Related U.S. Application Data
(60) Provisional application No. 63/620,006, filed on Jan. 11, 2024.

(51) Int. Cl. A61B 18/14; A61B 18/00

(52) U.S. Cl. CPC A61B 18/1492; A61B 2018/00077; A61B 2018/00083; A61B 2018/00482; A61B 2018/00595; A61B 2018/00601; A61B 2018/00607; A61B 2018/00916; A61B 2018/00982

(57) ABSTRACT

An endoscope treatment tool can include a sheath, a conductive first rod protruding from a distal end of the sheath, an insulator fixed to a distal end of the first rod, and a conductive second rod extending to a distal side from the insulator. The first rod and second rod are conductive, and the second rod is fixed in a position relative to the insulator along a longitudinal axis of the first rod.

### CROSS-REFERENCE TO RELATED APPLICATIONS

The present application claims priority based on U.S. Patent Provisional Application No. 63/620,006 filed in the United States on Jan. 11, 2024, the contents of which are incorporated herein by reference.

### TECHNICAL FIELD

This disclosure relates to an endoscope treatment tool.

### BACKGROUND

In endoscope treatments such as ESD (endoscopic submucosal dissection), as shown in PCT International Publication No. WO 2014/061701 (Patent Document 1) and the like, endoscope treatment tools such as high-frequency knives can be used. A surgeon can use endoscope treatment tools such as high-frequency knives to perform incisions of biological tissues.

### SUMMARY

When performing ESD procedures, the treatment tool must be frequently replaced depending on the purpose of the treatment. For this reason, a high-frequency knife described in Patent Document 1 and the like is required to be multifunctional so that it can easily perform tissue marking in addition to tissue incision without replacing the treatment tool.

The present disclosure provides an endoscope treatment tool that can perform a variety of treatments without replacing the treatment tool.

An endoscope treatment tool according to a first aspect of the present disclosure can include a sheath, a conductive first rod protruding from a distal end of the sheath, an insulator fixed to a distal end of the first rod, and a conductive second rod extending to a distal side from the insulator. The second rod can be fixed in a position relative to the insulator along a longitudinal axis of the first rod.

The endoscope treatment tool of the present disclosure can perform a variety of treatments without replacing the treatment tool.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an example of an overall view of an endoscope treatment system according to a first embodiment.

FIG. 2 is an example of an overall view showing a treatment tool of the endoscope treatment system.

FIG. 3 is an example of a perspective view of the distal end of the treatment tool.

FIG. 4 is an example of a side view of the distal end of the treatment tool.

FIG. 5 is an example of a side view of the distal end of the treatment tool.

FIG. 6 is an example of a cross-sectional view of the distal end of the treatment tool.

FIG. 7 is an example of a view showing an operating portion of the treatment tool.

FIG. 8 is an example of a view showing a switch of the operating portion.

FIG. 9 is an example of a view showing an incision and dissection step.

FIG. 10 is an example of a view showing an incision and dissection step.

FIG. 11 is an example of a view showing a modified first rod of the treatment tool.

FIG. 12 is an example of a view showing a modified first electrode and a modified insulating chip of the treatment tool.

FIG. 13 is an example of a view showing a modified first electrode and a modified insulating chip of the treatment tool.

FIG. 14 is an example of a cross-sectional view showing a modified switch.

FIG. 15 is an example of a cross-sectional view showing a modified operating wire and a modified connector of the treatment tool.

FIG. 16 is an example of a cross-sectional view showing the modification of the operating wire and the modification of the connector.

FIG. 17 is an example of a cross-sectional view showing another modification of the operating wire.

FIG. 18 is an example of a cross-sectional view showing the modification of the operating wire.

FIG. 19 is an example of a view showing the operating portion of the treatment tool according to the second embodiment.

FIG. 20 is an example of a cross-sectional view of the treatment tool when the first rod is retracted.

FIG. 21 is an example of a cross-sectional view of the treatment tool when the first rod is protruded.

FIG. 22 is an example of a view showing a modification of the receiving portion of the operating portion and a modification of the movable portion of the operating portion.

FIG. 23 is an example of a view showing a modification of the receiving portion and a modification of the movable portion.

FIG. 24 is an example of a view showing another modification of the receiving portion.

FIG. 25 is an example of a view showing another modification of the receiving portion and a modification of the movable portion.

FIG. 26 is an example of a view showing another modification of the receiving portion and a modification of the movable portion.

FIG. 27 is an example of a view showing a modification of the receiving portion and a modification of the movable portion.

FIG. 28 is an example of a view showing a modification of the receiving portion and a modification of the movable portion.

FIG. 29 is an example of a view showing another modification of the receiving portion.

### DETAILED DESCRIPTION

### First Embodiment

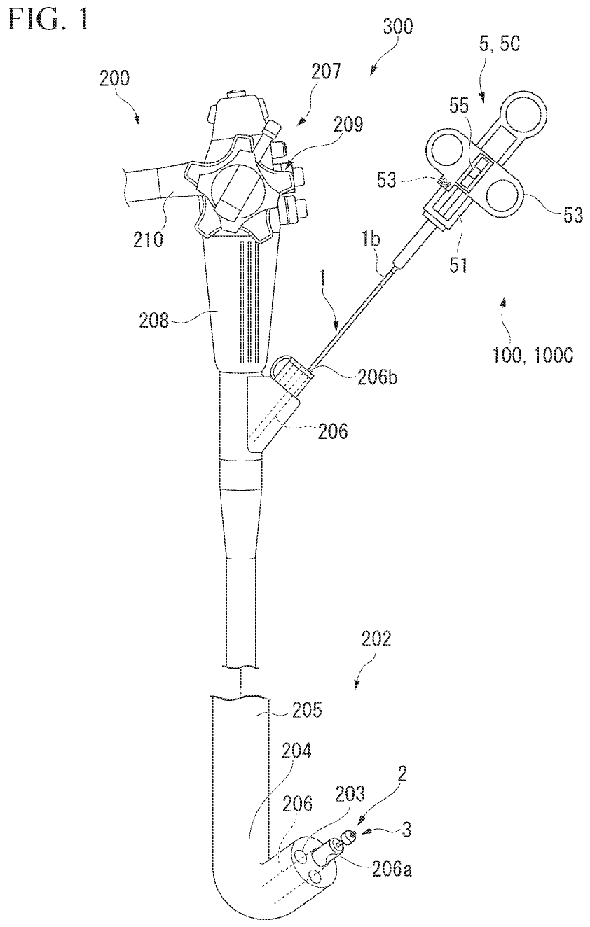

An endoscope treatment system 300 according to a first embodiment of the present disclosure will be described with reference to FIGS. 1 to 9. FIG. 1 is an example of an overall view of the endoscope treatment system 300 according to this embodiment.

### [Endoscope Treatment System 300]

As shown in FIG. 1, the endoscope treatment system 300 can include an endoscope 200 and a treatment tool 100. The treatment tool 100 can be configured to be inserted into the endoscope 200 for use.

### [Endoscope 200]

The endoscope 200 can be a flexible endoscope and can include an insertion portion 202 that is configured to be inserted into the body of a patient from the distal end, and an operating portion 207 attached to the proximal end of the insertion portion 202.

The insertion portion 202 can include an imaging portion 203, a bending portion 204, and a flexible portion 205. The imaging portion 203, the bending portion 204, and the flexible portion 205 can be arranged in this order from the distal end of the insertion portion 202. Inside the insertion portion 202, a channel 206 can be provided for inserting the treatment tool 100. A distal end opening 206a of the channel 206 can be provided at the distal end of the insertion portion 202.

The imaging portion 203 can be equipped with an imaging element such as a charge-coupled device (CCD), a complementary metal oxide semiconductor (CMOS), or any similar imaging device capable of capturing an image of the area to be treated. The imaging portion 203 can capture an image of the distal end of the treatment tool 100 when the treatment tool 100 protrudes from the distal end opening 206a of the channel 206.

The bending portion 204 can bend in response to the operation of the operating portion 207 by a user such as a surgeon. The flexible portion 205 can be a flexible tubular portion.

The operating portion 207 can be connected to the flexible portion 205. The operating portion 207 can include a grip 208, an input portion 209, a proximal end opening 206b of the channel 206, and a universal cord 210. The grip 208 can be a portion that is held by the surgeon (e.g., a handle). The input portion 209 can accept operation input for bending the bending portion 204.

The universal cord 210 can include a video signal line that outputs the image captured by the imaging portion 203 to the outside. The video signal line can be connected to a display device such as a liquid crystal display (LCD) via an image processing device equipped with a processor or the like.

### [Treatment Tool 100]

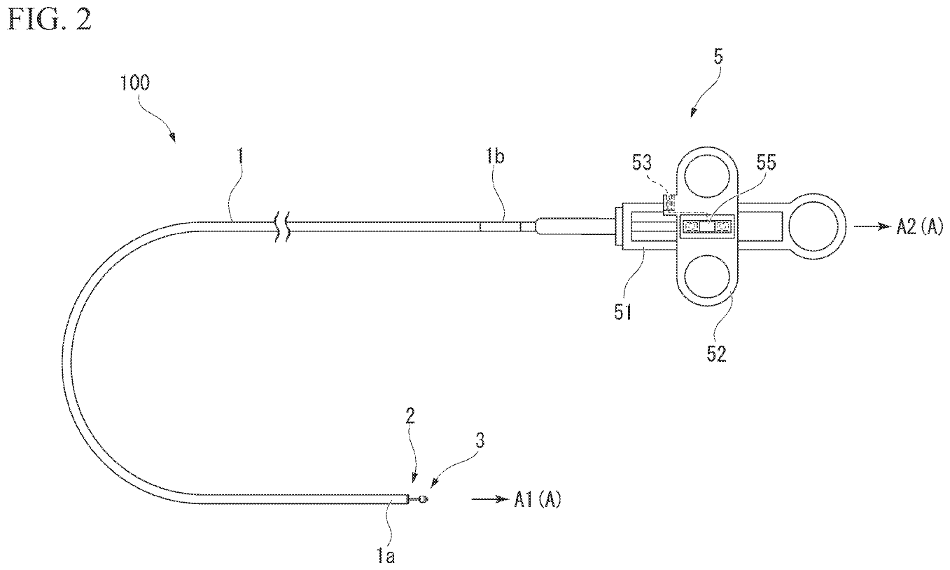

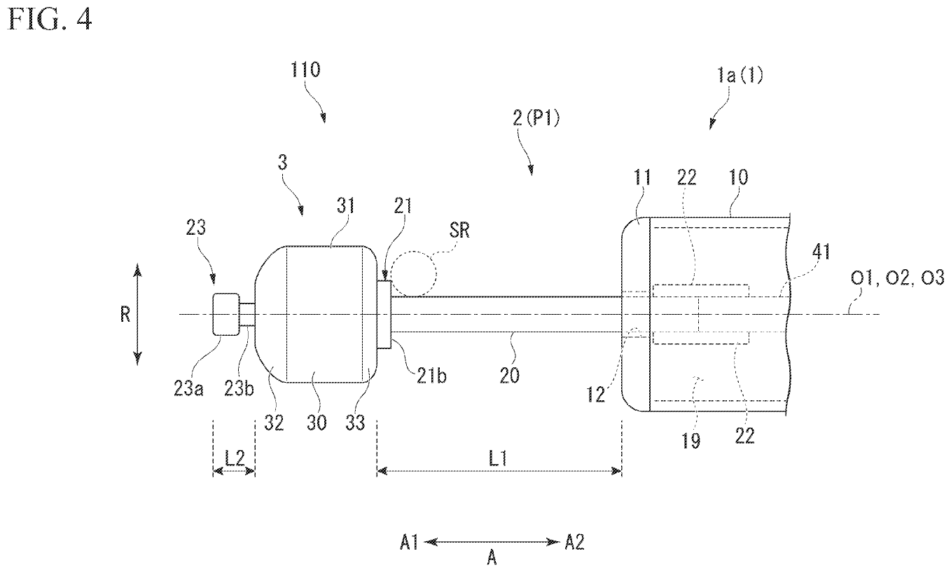

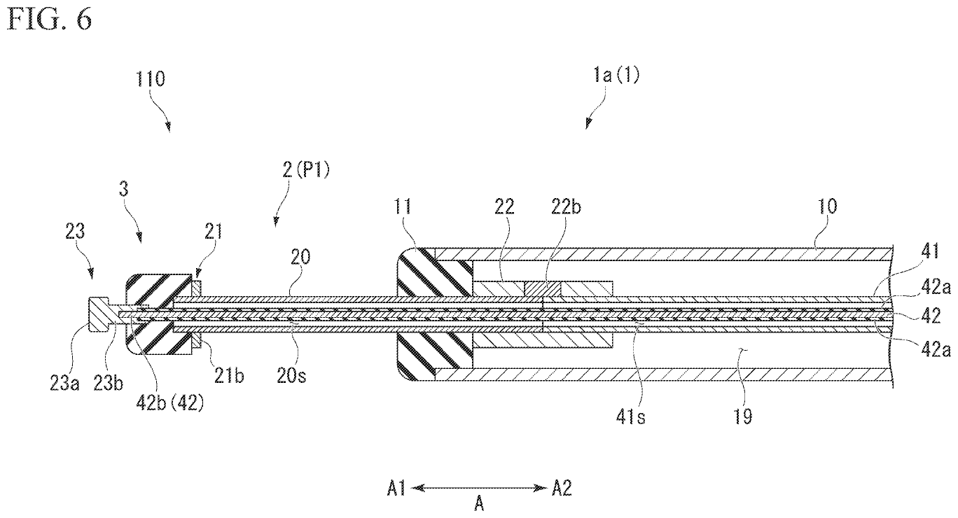

FIG. 2 is an example of an overall view showing the treatment tool 100. The treatment tool (endoscope treatment tool, high-frequency treatment tool) 100 can be an ESD knife. The treatment tool 100 can include a sheath 1, a knife 2, an insulating chip 3, an operating wire 41 (see FIG. 4), an electric wire (second wire) 42 (see FIG. 6), and an operating portion 5. The knife 2 and the insulating chip 3 can constitute a “treatment portion 110” that treats an affected area. In the following description, in the longitudinal direction (longitudinal direction, axial direction) A of the treatment tool 100, the side inserted into the patient's body is referred to as the “distal end side A1” (distal side A1) and the operating portion 5 side is referred to as the “proximal end side A2” (proximal side A2).

The sheath 1 can be or include a long tubular member extending from the distal end 1a to the proximal end 1b. The sheath 1 can be inserted into the channel 206 of the endoscope 200 and can advance and retract through the channel 206. As shown in FIG. 1, when the sheath 1 is inserted into the channel 206, the distal end 1a of the sheath 1 can protrude and retract from the distal end opening 206a of the channel 206.

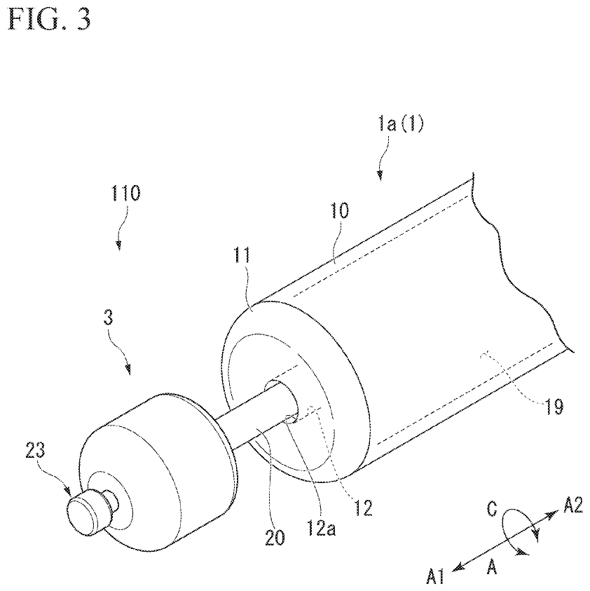

FIG. 3 is an example of a perspective view of the distal end of the treatment tool 100. The sheath 1 can include an outer tube 10 extending in the longitudinal direction A, and a distal end member 11 provided at the distal end of the outer tube 10. The sheath 1 can be formed by integrally molding the outer tube 10 and the distal end member 11.

The distal end member 11 can be formed in a cylindrical shape. Note that “cylindrical” includes shapes close to a cylindrical shape (e.g., substantially cylindrical) in addition to a strictly cylindrical shape. The distal end member 11 can be formed of an insulating material such as resin. The resin forming the distal end member 11 can include, for example, PTFE, PEEK, or ceramic such as zirconia. The distal end member 11 can include a through hole 12 formed therein.

The through hole 12 can be a hole provided in the distal end member 11 and can pass through the distal end member 11 in the longitudinal axis direction A. The distal end of the through hole 12 can communicate or engage with a first opening 12a formed in the distal end surface 14 of the distal end member 11. The proximal end of the through hole 12 can communicate or engage with the internal space 19 of the outer tube 10.

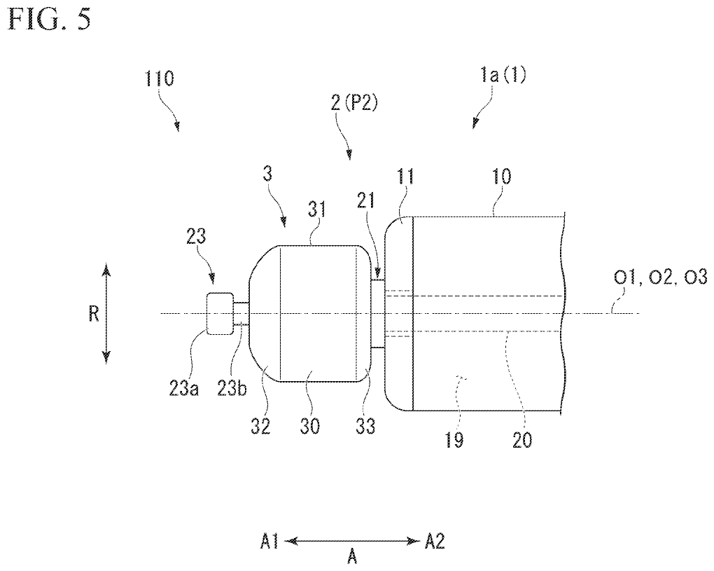

FIGS. 4 and 5 are examples of side views of the distal end of the treatment tool 100. The knife (electrode) 2 can be a metal member. The knife 2 can be made of a material such as stainless steel. The knife 2 can be conductive and can be energized with high-frequency current. The knife 2 can include a first rod 20, a first electrode 21, a connector 22, and a second rod 23.

The central axis O2 of the knife 2 in the longitudinal axis direction A can coincide or match with the central axis O1 of the sheath 1 in the longitudinal axis direction A. Note that “matching” or “coinciding” includes not only a strictly matching or coinciding state but also a nearly matching or coinciding state (e.g., substantially matching or coinciding).

FIG. 6 is an example of a cross-sectional view of the distal end of the treatment tool 100. The first rod (blade, electrode body) 20 can be a conductive metal round bar-shaped member. Note that “round bar-shaped” includes not only a strictly round bar shape but also a shape close to (e.g., substantially) a round bar shape. The first rod 20 can be hollow and can include an internal space 20s that penetrates the first rod 20 in the longitudinal axis direction A. The first rod 20 can be disposed on the distal end side A1 of the sheath 1. The operating wire 41 can be attached to the proximal end of the first rod 20.

The first rod 20 can be inserted through the through hole 12 of the distal end member 11 of the sheath 1 along the longitudinal axis direction A and can freely protrude and retract from the first opening 12a to the distal end side A1. The first rod 20 can be fixed in a state where it protrudes from the first opening 12a to the distal end side A1 and cannot advance or retract.

The first electrode (flange, enlarged diameter portion) 21 can be a disc-shaped conductive member provided at the distal end of the first rod 20. The first electrode 21 is not limited to a disc shape, and can be formed in a radial shape extending radially outward from the central axis O2 of the rod 20 in the radial direction R. A flat proximal end surface (back surface) 21b can be formed on the proximal end side A2 of the first electrode 21. The proximal end surface 21 is not limited to a flat surface, and may be, for example, an uneven surface. The knife 2 does not need to have the first electrode 21.

The connector (connecting member) 22 can be a cylindrical member made of metal. The term “cylindrical” includes not only a strictly cylindrical shape but also a shape close to a cylindrical shape (e.g., substantially cylindrical). The connector 22 can be provided with a through hole 22b that penetrates in the radial direction R. The connector 22 can connect the first rod 20 and the operating wire 41. For example, the first rod 20 and the operating wire 41 can be connected by soldering using solder inserted from the through hole 22b.

The second rod (second electrode) 23 can be a metal member having electrical conductivity. The second rod 23 can extend from the insulating chip 3 to the distal end side A1. The proximal end of the proximal end 23b of the second rod 23 can be fixed to the insulating chip 3 by bonding or press-fitting while being connected to the electric wire 42. In such an example, the second rod 23 cannot advance or retract with respect to the insulating chip 3, and its position on the longitudinal axis of the first rod 20 is fixed. As shown in FIG. 4, the length L2 from the distal end of the insulating chip 3 to the distal end of the second rod 23 can be smaller than the protruding length L1 of the first rod 20 from the sheath 1 when the first rod 20 is protruded to the distal end side A1 to the maximum.

The second rod 23 can include a distal end portion (flange, enlarged diameter portion) 23a disposed on the distal end side A1, and a proximal end portion 23b disposed on the proximal end side A2. The distal end portion 23a and the proximal end portion 23b can be arranged and connected in the longitudinal axis direction A. The distal end portion 23a and the proximal end portion 23b can be formed in a cylindrical shape. The outer diameter of the distal end portion 23a can be larger than the outer diameter of the proximal end portion 23b. The distal end portion 23a can be flange-shaped (disk-shaped). The shape of the distal end portion 23a is not limited to a flange shape, and may be, for example, a triangular shape or a hook shape.

The outer diameter of at least a part of the second rod 23 can be equal to or smaller than the outer diameter of the first rod 20. In this embodiment, the outer diameter of the proximal end portion 23b of the second rod 23 is smaller than the outer diameter of the first rod 20. The outer diameter of the distal end portion 23a of the second rod 23 can be smaller than the outer diameter of the first rod 20.

The first rod 20 and the first electrode 21, and the second rod 23 can be provided on either side of the insulating chip 3 in the longitudinal axis direction A and can be provided electrically separated.

A high-frequency current can be supplied to the first rod 20 and the first electrode 21 from an operating wire (first wire) 41 connected to the operating portion 5. When a high-frequency current is supplied to the first rod 20 and the first electrode 21 from the operating wire 41, the first rod 20 and the first electrode 21 can function as monopolar electrodes that output the high-frequency current to the biological tissue.

A high-frequency current can be supplied to the second rod 23 from an electric wire (second wire) 42 connected to the operating portion 5. When a high-frequency current is supplied to the second rod 23 from the electric wire 42, the second rod 23 can function as a monopolar electrode that outputs the high-frequency current to the biological tissue.

As shown in FIG. 4, when the knife 2 is advanced relative to the sheath 1, the distal end of the connector 22 can come into contact with the distal end member 11. The contact between the distal end of the connector 22 and the distal end member 11 can position the knife 2 at the first position P1, which is the position on the most distal side A1.

As shown in FIG. 5, when the knife 2 is retracted relative to the sheath 1, the proximal end surface 21b of the first electrode 21 can come into contact with the distal end surface 14 of the distal end member 11. The contact between the proximal end surface 21b of the first electrode 21 and the distal end member 11 can position the knife 2 at the second position P2, which is the position on the most proximal side A2.

As the operating wire 41 advances and retracts, the knife 2 can advance and retract from the first position P1 to the second position P2. The knife 2 can be fixed so as not to advance and retract in a state where it protrudes from the first opening 12a to the distal end side A1.

As shown in FIG. 4, by moving the knife 2 closer to the first position P1, a rear space SR can be secured on the proximal end side A2 of the first electrode 21. By securing the rear space SR of the first electrode 21, the surgeon can perform an incision procedure using the first electrode 21 in an appropriate manner.

The insulating chip (insulator) 3 can be made of an insulating material such as ceramic or resin. The insulating chip 3 can be provided on the distal end side A1 of the first electrode 21. In this embodiment, the proximal end of the insulating chip 3 is fixed to the distal end of the first rod 20 in contact with the first electrode 21. In a front view seen from the direction along the longitudinal axis A, the outer periphery of the first electrode 21 can be positioned radially inward of the outer surface 31 of the insulating chip 3 over the entire circumference. The insulating chip 3, the first rod 20, and the second rod 23 can be advanced and retracted as a unit relative to the sheath 1.

The central axis O3 of the insulating chip 3 in the longitudinal axis direction A can coincide with the central axis O1 of the sheath 1 in the longitudinal axis direction A. Incidentally, “coinciding” includes a state in which they coincide strictly as well as a state in which they almost or substantially coincide.

The insulating chip 3 can include a main body 30, a distal end portion 32 disposed on the distal end side A1 of the main body 30, and a proximal end portion 33 disposed on the proximal end side A2 of the main body 30. The distal end portion 32, the main body 30, and the proximal end portion 33 can be arranged and connected in the longitudinal axis direction A.

The main body 30 can be formed in a cylindrical shape. The central axis of the distal end portion 32 in the longitudinal axis direction A can coincide with the central axis O3.

The distal end portion 32 can be provided at the distal end of the main body portion 30. The distal end portion 32 can be formed in a hemispherical (dome) shape, and the diameter tapers toward the distal end side A1.

The proximal end portion 33 can be provided at the proximal end of the main body portion 30. The proximal end portion 33 can be formed in a hemispherical (dome) shape, and the diameter tapers toward the proximal end side A2.

The operating wire 41 can be a metal wire that passes through the internal space (pipe, lumen) 19 of the outer tube 10. The operating wire 41 can be formed of a material such as stainless steel. The operating wire 41 can be hollow, and can include an internal space 41s that passes through the operating wire 41 in the longitudinal axis direction A. The distal end of the operating wire 41 can be connected to the first rod 20, and the internal space 41s of the operating wire 41 can communicate or engage with the internal space 20s of the first rod 20. The proximal end of the operating wire 41 can be connected to the operating portion 5.

The electric wire 42 can be a metal wire. The electric wire 42 can pass through the internal space 41s of the operating wire 41 and the internal space 20s of the first rod 20. An insulating coating 42a can be provided on the outer periphery of the electric wire 42, and the electric wire 42 can be insulated from the operating wire 41 and the first rod 20. The insulating coating 42a can be removed from the distal end 42b of the electric wire 42, and the distal end 42b can be connected to the second rod 23 by brazing or the like.

The electric wire 42 can be thickened to increase its rigidity so that it can be advanced and retracted, and the second rod 23 can be advanced and retracted relative to the insulating chip 3 and the first rod 20 in response to the advancement and retract of the electric wire 42.

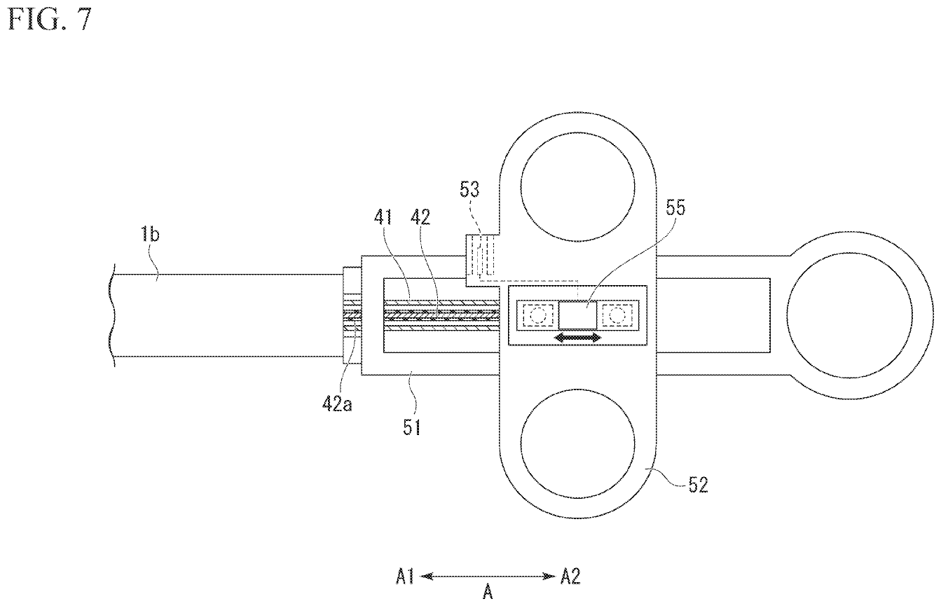

FIG. 7 shows an example of the operating portion 5. The operating portion 5 can include an operating portion main body 51, a slider 52, a power supply connector 53, and a switch 55.

The distal end of the operating portion main body 51 can be connected to the proximal end 1b of the sheath 1. The operating portion main body 51 can include an internal space through which the operating wire 41 can be inserted. The operating wire 41 can pass through the internal space 19 of the outer tube 10 and the internal space of the operating portion main body 51 and can extend to the slider 52.

The slider (handle) 52 can be attached to the operating portion main body 51 so as to be movable along the longitudinal axis direction A. The proximal end of the operating wire 41 can be attached to the slider 52. The surgeon can advance and retract the slider 52 relative to the operating portion main body 51, thereby advancing and retracting the operating wire 41, the knife 2, and the insulating chip 3.

The power supply connector 53 can be fixed to the slider 52. The power supply connector 53 can be connected to a high-frequency power supply device via an electric cable. The power supply connector 53 can supply high-frequency current supplied from the high-frequency power supply device to the first rod 20 and the first electrode 21 via the operating wire 41. The power supply connector 53 can also supply high-frequency current supplied from the high-frequency power supply device to the second rod 23 via the electric wire 42. In another example, the power supply connector 53 can be fixed to the operating portion main body 51 instead of the slider 52.

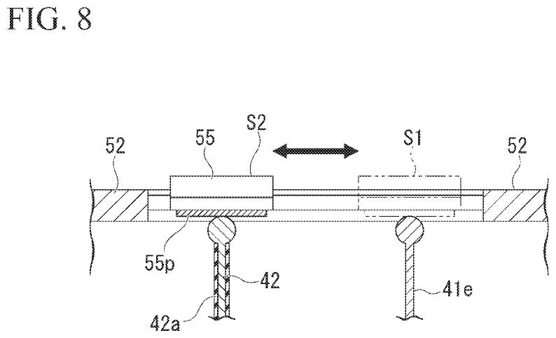

FIG. 8 is an example of a diagram showing the switch 55. The switch 55 can be provided on the slider 52. The switch 55 can be a switch that alternatively selects one of the operating wire 41 or the electric wire 42 as the current path and can include a connection pad 55p attached to it. The connection pad 55p and the power supply connector 53 can be connected by a metal wire or the like. In other words, the high-frequency current supplied from the power supply connector 53 is only passed through one of the wires selected by the switch 55.

The switch 55 can include a connection pad 55p electrically connected to the power supply connector 53. The switch 55 can slide between a first position S1 where the connection pad 55p is electrically connected to the conductive wire 41 and a second position S2 where the connection pad 55p is electrically connected to the electric wire 42. The conductive wire 41 can be a wire electrically connected to the operating wire 41. In an example, conductive wire 41e may not essential, and the operating wire 41 may extend to the connection pad 55p. In that case, when the switch 55 is located at the first position S1, the operating wire 41 is electrically connected directly to the connection pad 55p.

When the switch 55 is in the first position S1, the high-frequency current supplied from the power supply connector 53 can be supplied to the first rod 20 and the first electrode 21 via the operating wire 41. When the switch 55 is in the second position S2, the high-frequency current supplied from the power supply connector 53 can be supplied to the second rod 23 via the electric wire 42.

### [Method of Using the Endoscope Treatment System 300]

Next, a procedure (method of using the endoscope treatment system 300) using the endoscope treatment system 300 of this embodiment will be described. Specifically, the incision and dissection of the lesion in endoscope treatment such as ESD (endoscopic submucosal dissection) will be described.

As a preparatory step, the surgeon can identify the lesion using any desired or appropriate method. For example, the surgeon can insert the insertion portion 202 of the endoscope 200 into the digestive tract (e.g., esophagus, stomach, duodenum, large intestine) and identify the lesion while observing the image obtained by the imaging portion 203 of the endoscope.

### <Insertion Step>

The surgeon can insert the treatment tool 100 into the channel 206 and protrude the distal end 1a of the sheath 1 from the distal end opening 206a of the insertion portion 202. The surgeon can advance the slider 52 of the operating portion 5 relative to the operating portion main body 51, protruding the knife 2 and the insulating chip 3.

### <Marking Step>

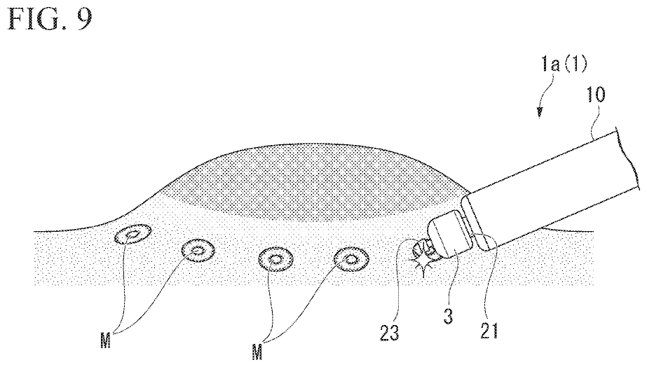

FIG. 9 shows an example of the marking step. The surgeon can retract the slider 52 of the operating portion 5 relative to the operating portion body 51 and place the knife 2 at the proximal end side A2 position (e.g., second position P2). With the second rod 23 in contact with the mucosal surface, the surgeon can move the switch 55 to the second position S2 to energize the second rod 23, thereby cauterizing and coagulating the biological tissue (mucosal surface) around the lesion. As a result, marking M can be applied to the mucosal surface. The surgeon can also cauterize and coagulate the biological tissue (mucosal surface) using the first electrode 21. The marking M on the biological tissue differs from an incision in that the tissue is denatured by coagulating the biological tissue (mucosal surface), and therefore the marking M is different from incision.

### <Incision and Dissection Step>

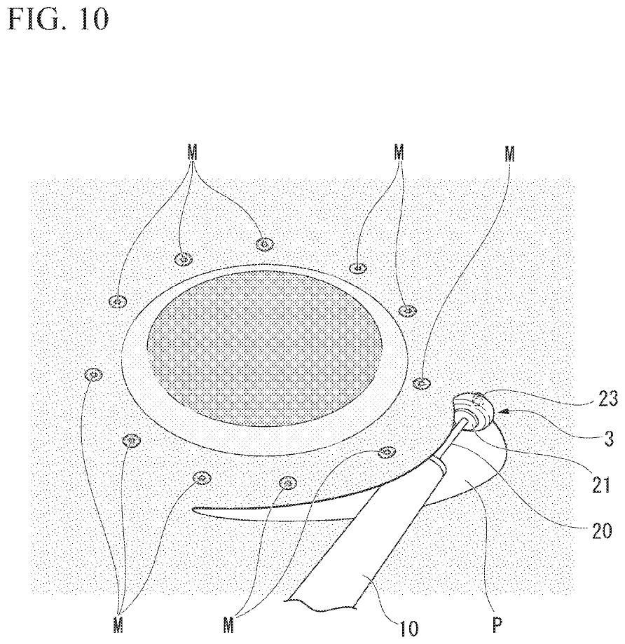

FIG. 10 shows an example the incision and dissection step. The surgeon can perform the incision and dissection procedure. With the second rod 23 in the state of being inserted under the mucosal layer, the surgeon can make a pre-cut (an entry point for starting the incision) P in the mucosal layer and/or submucosal layer by moving the switch 55 to the second position S2 and passing electricity through the second rod 23.

The surgeon can advance the slider 52 of the operating portion 5 relative to the operating portion body 51 and place the knife 2 at the position of the distal end side A1 (for example, the first position P1). With the switch 55 moved to the first position S1 to pass a high-frequency current through the first rod 20, the surgeon can move the first rod 20 from the pre-cut P to incise the mucosa of the lesion. With a high-frequency current passing through the first rod 20, as shown in FIG. 10, the surgeon can lift the mucosa of the incised lesion to expose the submucosa, and peel off the submucosa of the incised lesion. At this time, because no high-frequency current is passed through the second rod 23, the second rod 23 does not incise tissues such as the mucosa or submucosa.

The surgeon can continue the above-mentioned operation (treatment) as necessary, and finally excise the lesion and completes the ESD procedure.

The treatment tool 100 according to this embodiment allows the surgeon to easily perform marking on the biological tissue in addition to incising it without replacing the treatment tool. The surgeon can supply high-frequency current to only one of the first rod 20 and the second rod 23 by operating the switch 55 according to the treatment to be performed.

The first embodiment of the present disclosure has been described above in detail with reference to the drawings, but the specific configuration is not limited to this embodiment, and design changes and the like that do not deviate from the gist of the present disclosure are also included. In addition, the components shown in the above-mentioned embodiment and modifications can be appropriately combined to configure the present disclosure.

### Modification 1

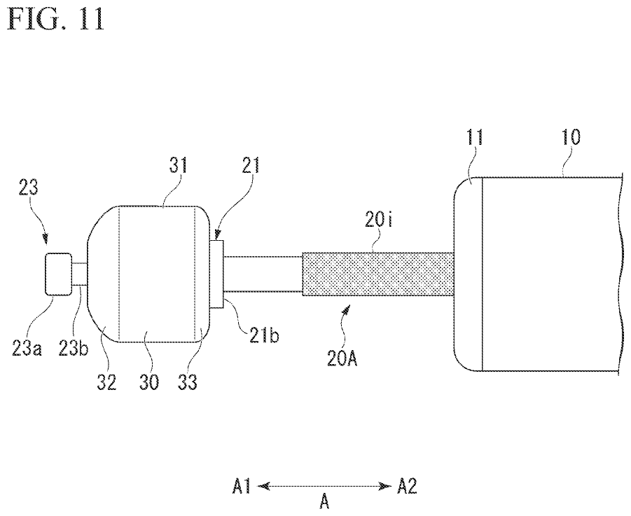

FIG. 11 shows an example of a modification of the first rod 20A of the first rod 20. The proximal end side of the first rod 20A protruding from the sheath 1 can be covered with an insulating member 20i. The insulating member 20i can be, for example, an insulating coating or an insulating tube. Since electricity is not passed through the biological tissue from at least a part of the outer circumferential surface of the first rod 20A, thermal invasion of the biological tissue can be suppressed.

### Modification 2

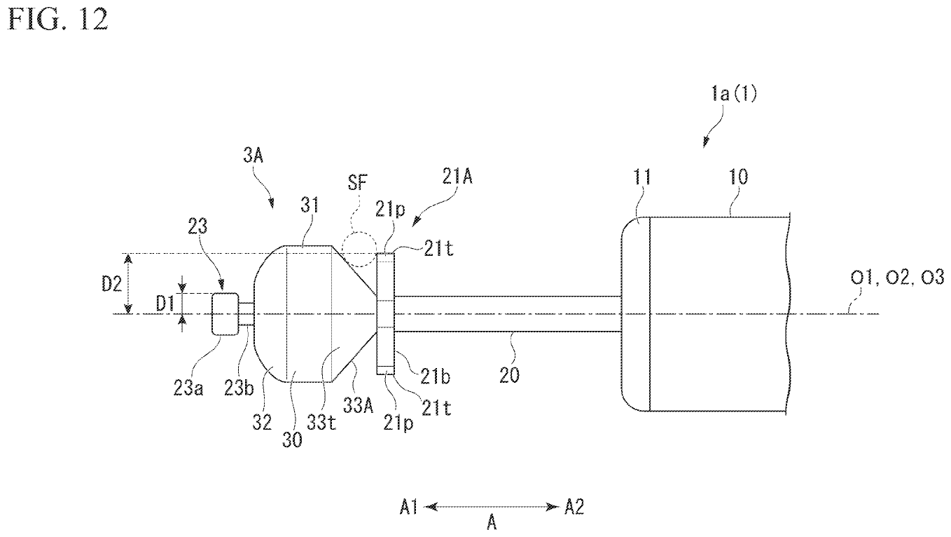

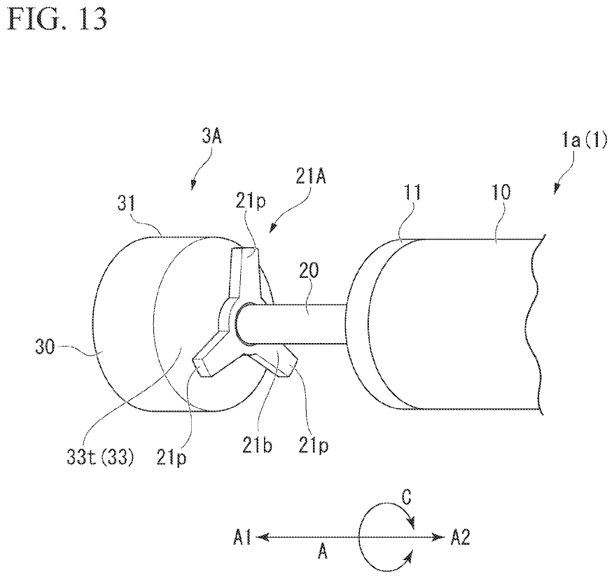

FIGS. 12 and 13 are examples of diagrams showing a first electrode 21A, which is a modification of the first electrode 21, and an insulating chip 3A, which is a modification of the insulating chip 3. The first electrode 21A can be a plate-shaped conductive member provided at the distal end of the first rod 20. The first electrode 21A can include a protruding portion 21p extending in a direction intersecting with the longitudinal axis of the first rod 20. For example, the protruding portion 21p can protrude outward in the radial direction R of the first rod 20 beyond the outer surface of the proximal end of the insulating chip 3A (the proximal end of the tapered surface 33t described later). A planar proximal end surface (back surface) 21b can be formed on the proximal end side A2 of the first electrode 21A. The proximal end surface 21b is not limited to a planar surface, and may be, for example, an uneven surface.

The first electrode 21A can include multiple protrusions such as the three protrusions 21p, shown. The three protrusions 21p can be arranged at equal intervals along the circumferential direction C relative to the longitudinal axis direction A. In a front view from the direction along the longitudinal axis direction A, the first electrode 21A can be formed in a three-pronged shape. The first electrode 21A can also be formed in a four- or five-pronged shape. That is, the first electrode 21A can be formed in a radial shape extending radially outward in the radial direction R from the central axis O2 of the rod 20. The first electrode 21A can also be formed in a flange shape such as a disk shape or a polygonal shape.

The insulating chip (insulator) 3A can be formed of an insulating material such as ceramic or resin. The insulating chip 3A can be provided on the distal end side A1 of the first electrode 21A. The proximal end of the insulating chip 3A can be fixed to the first rod 20 in a state of contact with the first electrode 21A. At least the protruding portion 21p of the first electrode 21A protrudes outward in the radial direction R beyond the outer circumferential surface of the proximal end of the insulating chip 3A (the proximal end of a tapered surface 33t described later).

In a front view from the longitudinal axis direction A, the maximum distance D1 from the central axis of the second rod 23 to the outer surface of the second rod 23 can be smaller than the distance D2 from the central axis of the first rod 20 to the top 21t of the protruding portion 21p of the first electrode 21A.

The insulating chip 3A can include a main body 30, a distal end portion 32, and a proximal end portion 33A which is a modified version of the proximal end portion 33. The proximal end portion 33A can include a tapered surface 33t which narrows toward the first electrode 21A (toward the proximal end side A2). The tapered surface 33t can be spaced apart from the protruding portion 21p with a gap therebetween, and a front space (e.g., a gap) SF can be formed between the tapered surface 33t and the protruding portion 21p. That is, in the longitudinal axis direction A of the first rod 20, the tapered surface 33t and the protruding portion 21p can be spaced apart with a gap and can be disposed so that the tapered surface 33t and the distal end surface of the protruding portion 21p face each other. By securing the space (gap) SF in front of the first electrode 21A, the surgeon can perform an incision procedure using the first electrode 21A.

### Modification 3

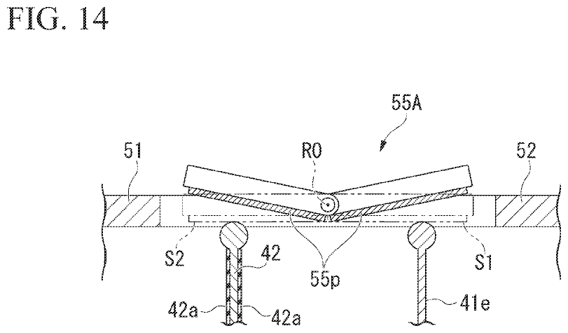

FIG. 14 is an example of a cross-sectional view showing a switch 55A, which is a modification of the switch 55. The switch 55A can be moved between a first position S1 where the connection pad 55p is connected to the conductive wire 41 and a second position S2 where the connection pad 55p is connected to the electric wire 42, not by sliding movement but by rotation about the rotation axis RO.

### Modification 4

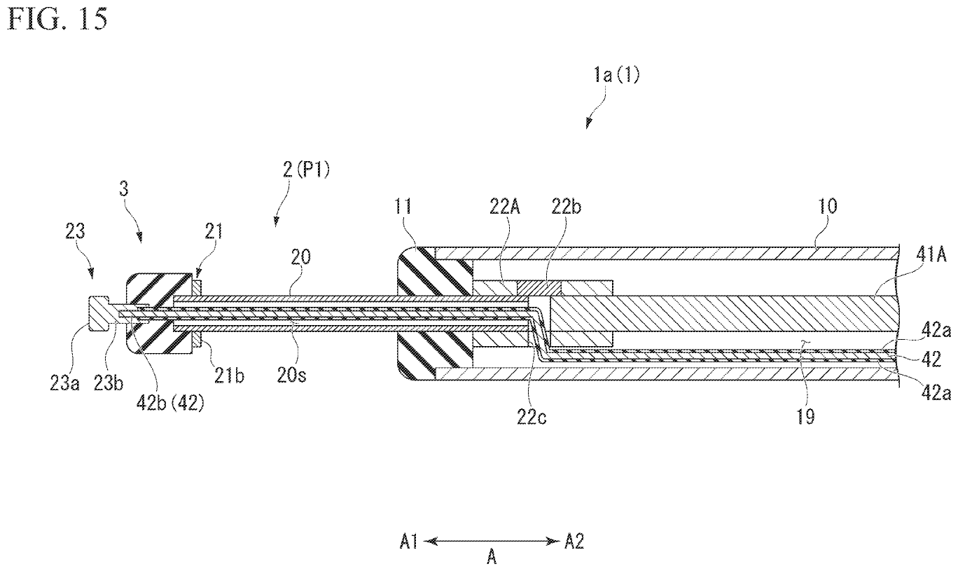

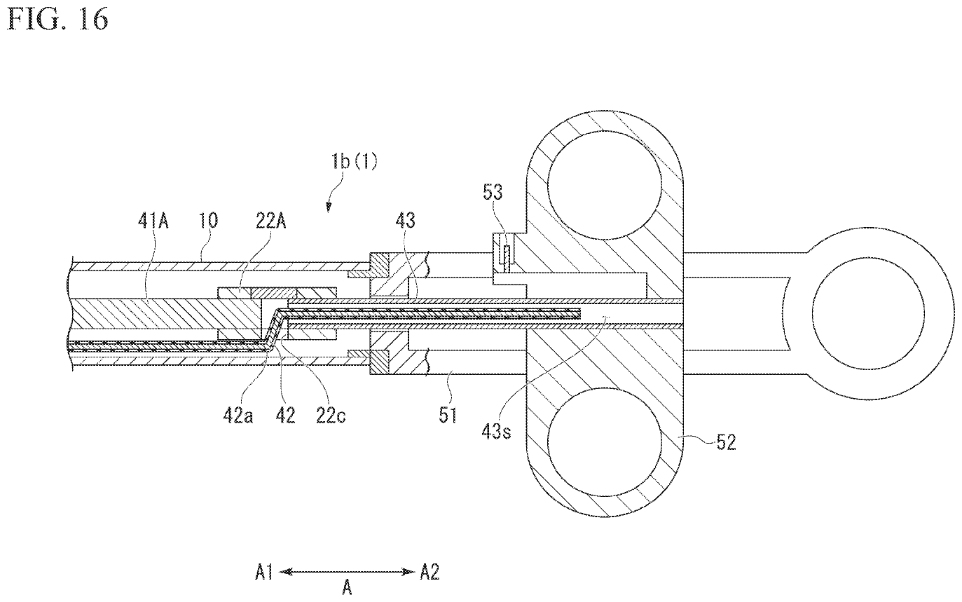

FIGS. 15 and 16 are examples of cross-sectional views showing an operating wire 41A, which is a modification of the operating wire 41, and a connector 22A, which is a modification of the connector 22. Unlike the hollow operating wire 41, the operating wire 41A is solid. The connector 22A can be provided with a through hole 22c penetrating in the radial direction R. The electric wire 42, which passes through the internal space 20s of the first rod 20, can pass through the through hole 22c of the connector 22A and pass through the internal space 19 of the outer tube 10.

The proximal end of the operating wire 41A can be connected to a hollow metal pipe 43 by the connector 22A. The high-frequency current supplied to the conductive wire 41 can be supplied to the first rod 20 and the first electrode 21 via the metal pipe 43 and the operating wire 41A.

The proximal end of the electric wire 42 can pass through the through hole 22c of the connector 22A and can pass through the internal space 43s of the metal pipe 43. The electric wire 42 can extend through the internal space 43s of the metal pipe 43 to the slider 52.

The electric wire 42 can be inserted through the brazing through hole 22b instead of the through hole 22c of the connector 22A. The through hole 22b can be blocked by pouring solder into it while the electric wire 42 is inserted through the through hole 22b. In this case, the internal space 19 of the sheath 1 and the internal space 20s of the rod 20 do not communicate with each other.

### Modification 5

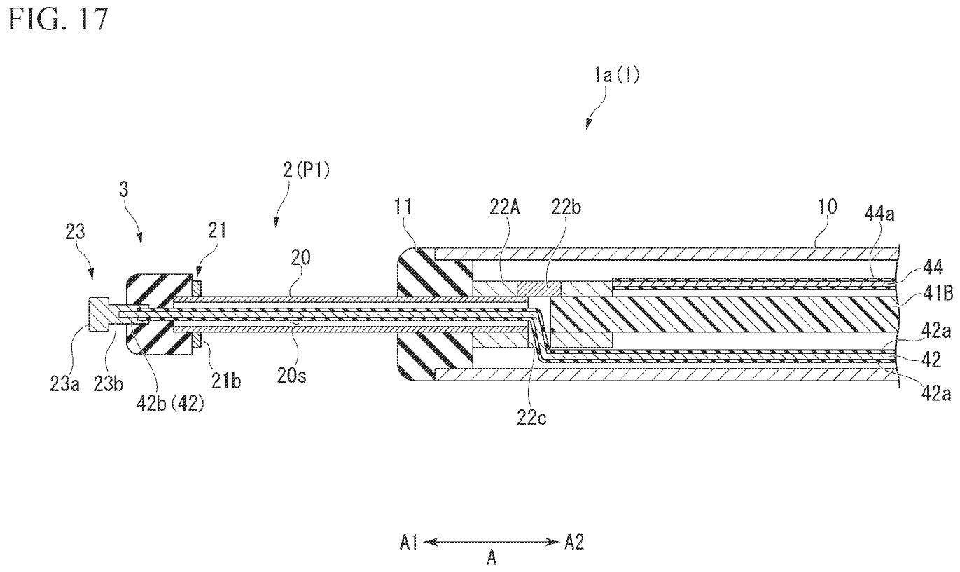

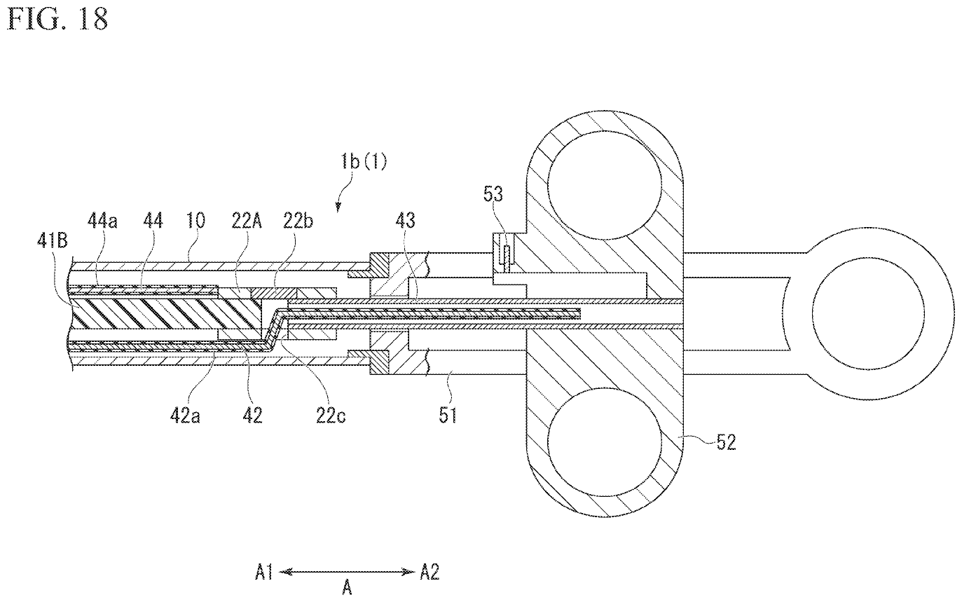

FIGS. 17 and 18 are examples of cross-sectional views showing an operating member 41B, which is a modification of the operating wire 41. The operating member 41B can be a long solid member that is made of resin or the like and as such can have no conductivity. The connector 22A on the distal end side A1 and the connector 22A on the proximal end side A2 can be electrically connected by a conductive wire 44. An insulating coating 44a can be provided on the outer periphery of the conductive wire 44. The high-frequency current supplied to the conductive wire 41e can be supplied to the first rod 20 and the first electrode 21 via the metal pipe 43 and the conductive wire 44.

### Second Embodiment

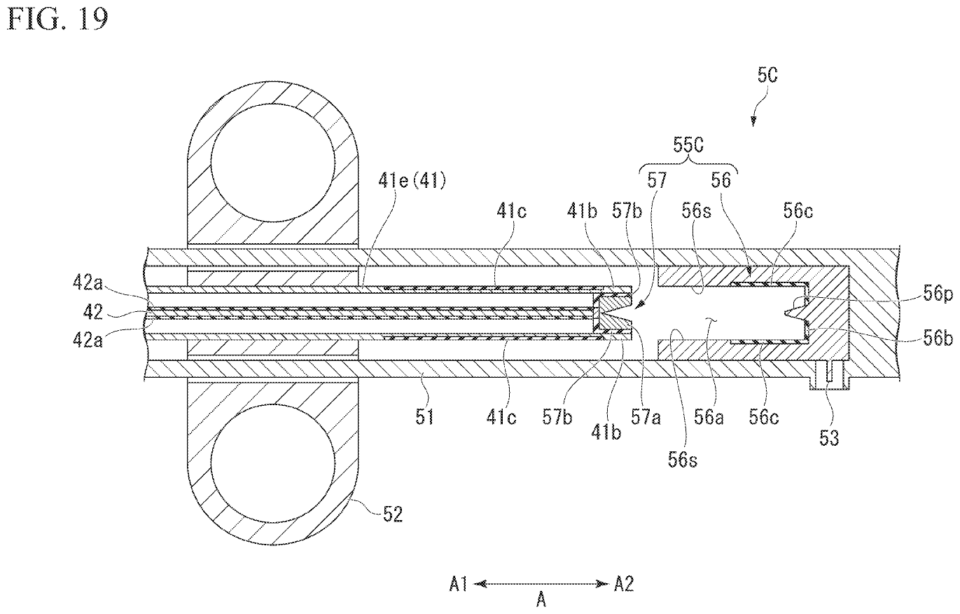

A treatment tool 100C according to a second embodiment of the present disclosure will be described with reference to FIGS. 19 to 21. In the following description, components common to those already described will be given the same reference numerals and a duplicate description will be omitted.

The treatment tool (endoscope treatment tool, high-frequency treatment tool) 100C can be an ESD knife. The treatment tool 100 can include a sheath 1, a knife 2, an insulating chip 3, an operating wire 41, an electric wire 42, and an operating portion 5C.

FIG. 19 is an example of a diagram showing the operating portion 5C. The operating portion 5C can include an operating portion main body 51, a slider 52, a power supply connector 53, and a switch 55C.

The switch 55C can be a switch that alternatively selects either the operating wire 41 or the electric wire 42 as the current path depending on the advancement and retract of the slider 52. The switch 55C can include a receiving portion 56 and a movable portion 57.

The receiving portion 56 can be a conductive member fixed to the operating portion main body 51 and can be electrically connected to the power supply connector 53. The receiving portion 56 can be formed with a recess 56a in which the movable portion 57 is housed. The recess 56a can open toward the distal end side A1 and can house the movable portion 57 that retracts toward the proximal end side A2. A plug 56p that protrudes toward the distal end side A1 can be provided on a bottom surface 56b formed on the proximal end side A2 of the recess 56a. The plug 56p can be formed in a cone shape. An insulating member 56c can be provided on the bottom surface 56b of the recess 56a other than the plug 56p and on the inner surface 56s of the recess 56a on the proximal end side A2.

The movable portion 57 can be a conductive member fixed to the proximal end of the operating wire 41 and can advance and retract along the longitudinal axis direction A in response to the advancement and retract of the slider 52. A plug receiving portion 57a can be formed on the end of the proximal end side A2 of the movable portion 57. The plug receiving portion 57a can be electrically connected to the electric wire 42. The plug receiving portion 57a can include a conical recess formed therein and can engage with the plug 56p. The plug 56p and the plug receiving portion 57a can engage with each other when the movable portion 57 is accommodated in the receiving portion 56. The plug receiving portion 57a can be covered with an insulating member 57b except for the part electrically connected to the electric wire 42 and the part that engages with the plug 56p.

The outer surface 41 of the operating wire 41 can include an insulating member 41c at its proximal end other than the most proximal end 41b. Here, the most proximal end 41b is the part of the proximal end of the outer surface 41 of the operating wire 41 that is closest to the proximal end side A2.

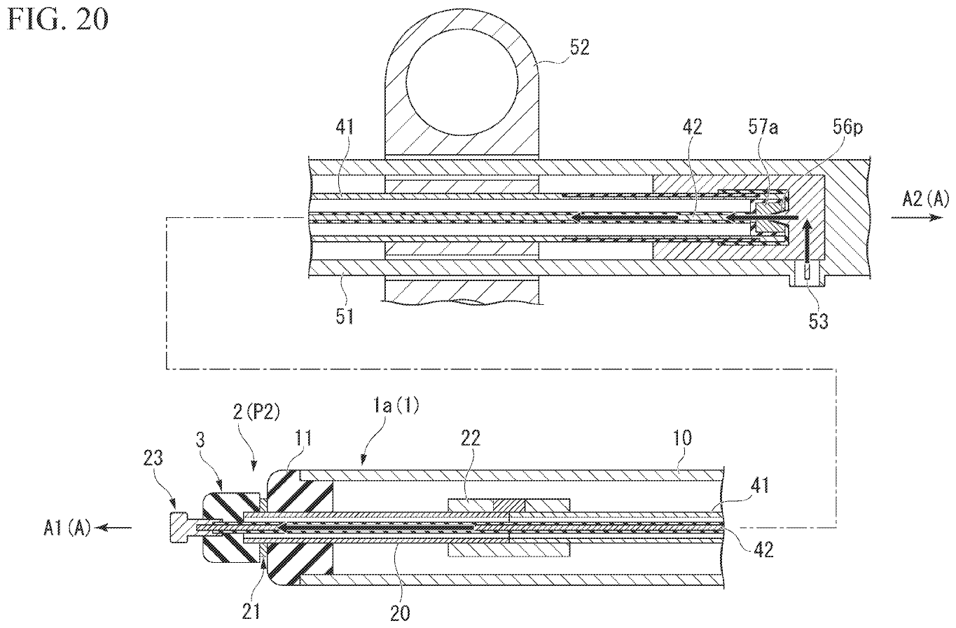

FIG. 20 is an example a cross-sectional view of the treatment tool 100C when the first rod 20 is retracted. The surgeon can move the slider 52 to the proximal end side A2 to retract the first rod 20 into the sheath 1. The movable portion 57 can be accommodated in the receiving portion 56, so that the plug 56p and the plug receiving portion 57a engage. The high-frequency current supplied from the power supply connector 53 can be supplied to the second rod 23 via the plug 56p, the plug receiving portion 57a, and the electric wire 42.

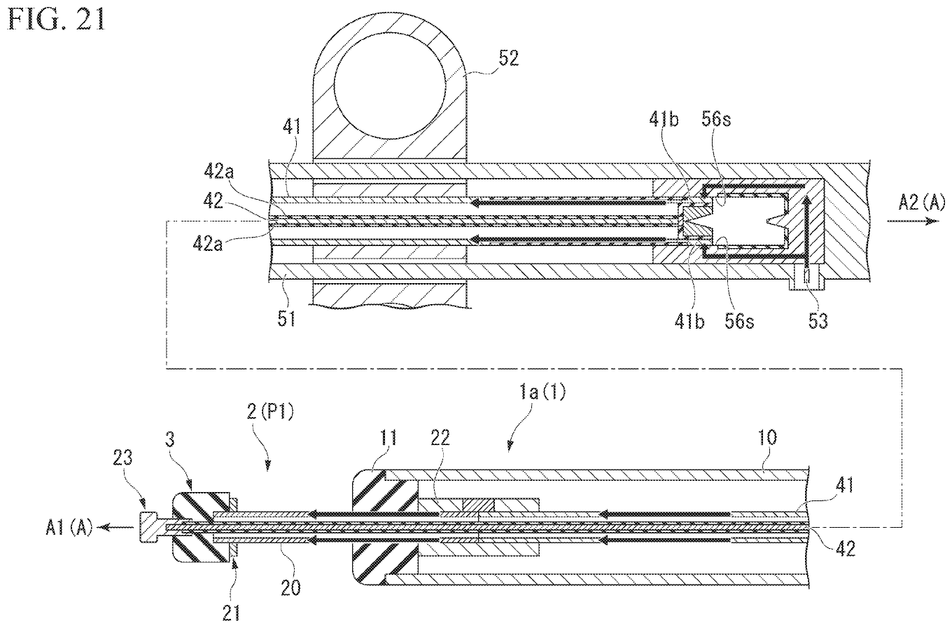

FIG. 21 is an example of a cross-sectional view of the treatment tool 100C when the first rod 20 is protruded. The surgeon can move the slider 52 to the distal end side A1 to project the first rod 20 from the sheath 1. The proximal end 41b of the control wire 41 can come into contact with the non-insulated inner surface 56s. The high-frequency current supplied from the power supply connector 53 can be supplied to the first rod 20 and the first electrode 21 via the inner surface 56s, the proximal end 41b, and the control wire 41.

With the treatment tool 100C according to this embodiment, in addition to incising the biological tissue, marking on the biological tissue can be easily performed without replacing the treatment tool. The surgeon can supply high-frequency current to only one of the first rod 20 and the second rod 23 depending on the advancement and retract of the insulating chip 3, the first rod 20, and the second rod 23 relative to the sheath 1. As shown in FIG. 20, the surgeon can supply high-frequency current only to the second rod 23 by moving the insulating chip 3, the first rod 20, and the second rod 23 to the proximal end side A2. As shown in FIG. 21, the surgeon can supply high-frequency current only to the first rod 20 and the first electrode 21 by moving the first rod 20 and the second rod 23 to the distal end side A1.

The second embodiment of the present disclosure has been described above in detail with reference to the drawings, but the specific configuration is not limited to this embodiment, and design changes and the like within the scope of the gist of the present disclosure are also included. In addition, the components shown in the above-mentioned embodiment and modifications can be appropriately combined to configure the present disclosure.

### Modification 2-1

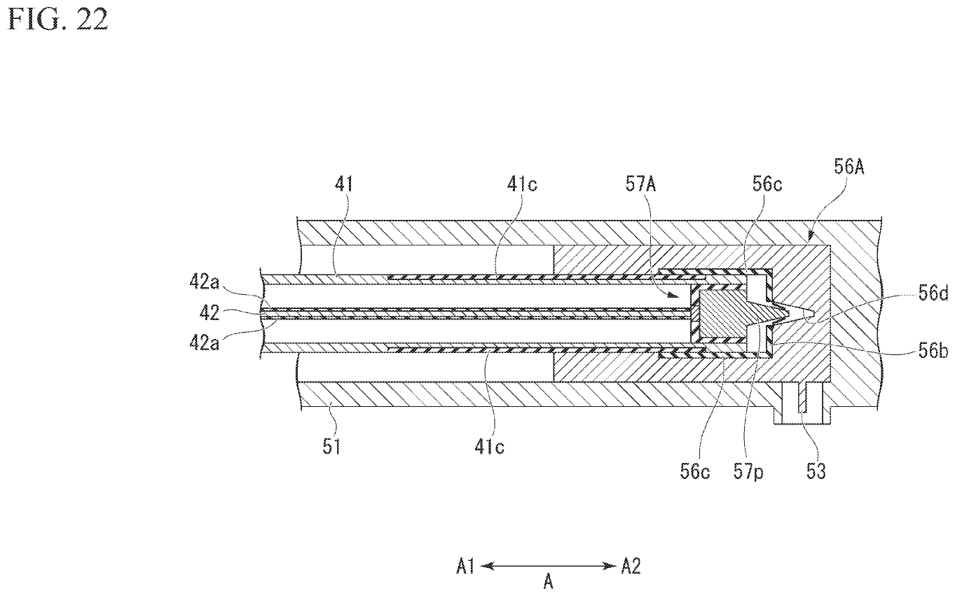

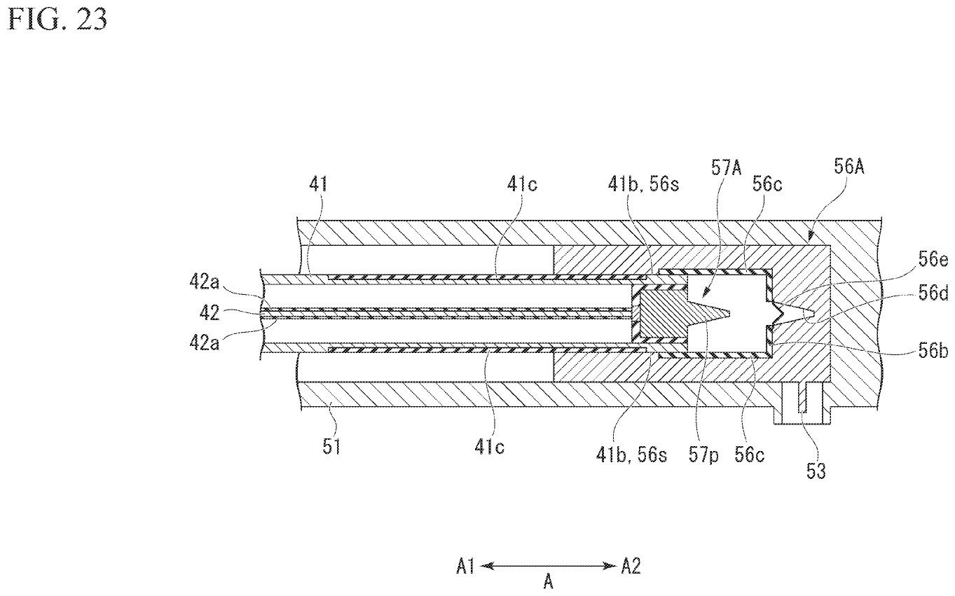

FIGS. 22 and 23 are example diagrams showing a receiving portion 56A, which is a modification of the receiving portion 56, and a movable portion 57A, which is a modification of the movable portion 57. A plug receiving portion 56d recessed into the proximal end side A2 can be provided on the bottom surface 56b of the receiving portion 56A. A plug 57p can be formed on the end of the proximal end side A2 of the movable portion 57A. When the movable portion 57A is accommodated in the receiving portion 56A, the plug 57p and the plug receiving portion 56d can engage with each other.

The plug receiving portion 56d can be provided with or include a leaf spring 56e. The plug 57p accommodated in the plug receiving portion 56d can be inserted while pushing the leaf spring 56e apart. This stabilizes the electrical contact between the plug 57p and the plug receiving portion 56d.

### Modification 2-2

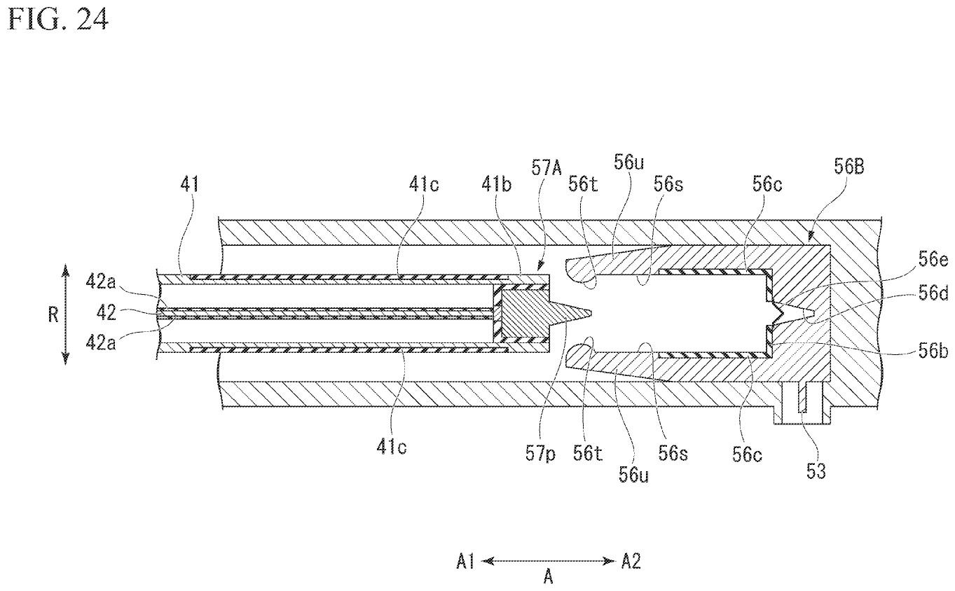

FIG. 24 shows an example of a receiving portion 56B, which is a modification of the receiving portion 56A. The receiving portion 56B can include a protrusion 56t that projects inward in the radial direction R on the inner surface 56s where the insulating member 56c is not provided. The protrusion 56t can be provided, for example, on the inner surface 56s at the most distal end side A1. The support portion 56u on which the protrusion 56t is provided can be formed in a cantilever shape and is elastically deformable in the radial direction R. When the movable portion 57A is inserted into the receiving portion 56B, the movable portion 57A can push and spread the protrusions 56t. This stabilizes the electrical contact between the most proximal end 41b and the inner surface 56s. In an example, the protrusions 56t can be ball plungers.

### Modification 2-3

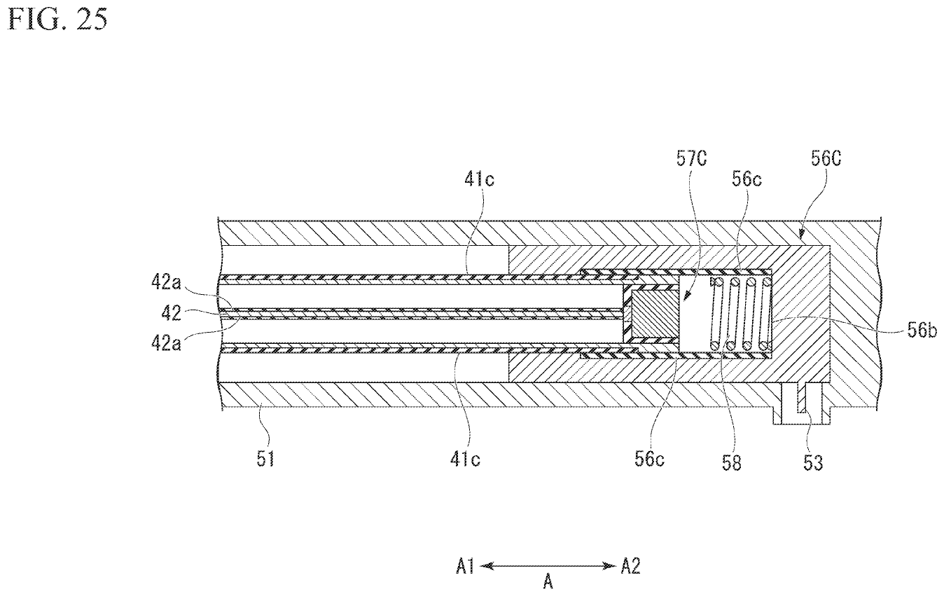

FIG. 25 shows an example of a receiving portion 56C, which is a modification of the receiving portion 56, and a movable portion 57C, which is a modification of the movable portion 57. In such an example, the receiving portion 56C and the movable portion 57C do not have the plug 56p and the plug receiving portion 57a. The bottom surface 56b of the receiving portion 56C and the movable portion 57C can come into contact with each other via a conductive spring 58. This widens the contactable range, so that the electrical contact between the bottom surface 56b of the receiving portion 56C and the movable portion 57C is stabilized.

### Modification 2-4

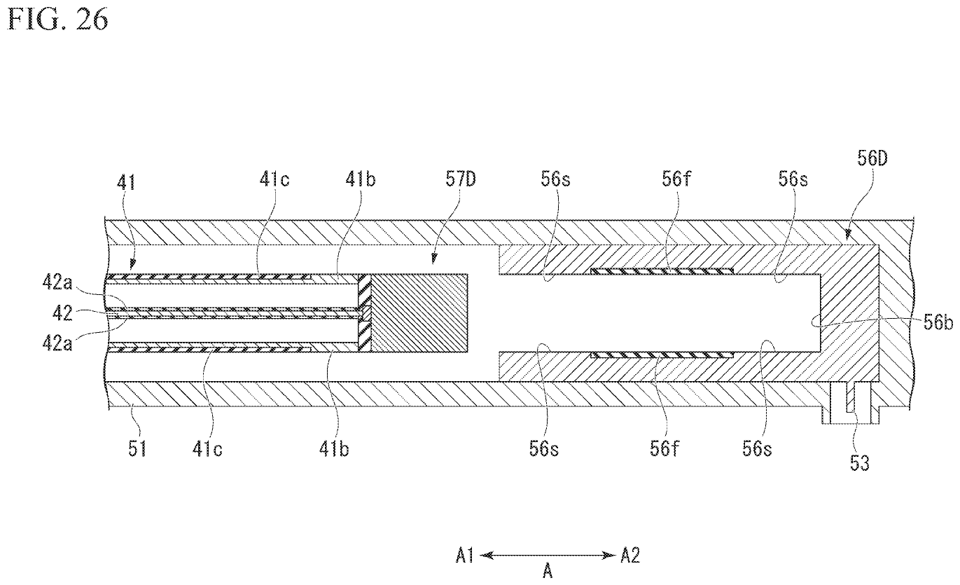

FIGS. 26 to 28 show examples of a receiving portion 56D, which is a modification of the receiving portion 56, and a movable portion 57D, which is a modification of the movable portion 57. The receiving portion 56D does not have a plug 56p. The movable portion 57D can be formed in a cylindrical shape and does not have a plug receiving portion 57a or an insulating member 57b. The movable portion 57D can be electrically connected to the receiving portion 56D by contacting the inner surface 56s and the bottom surface 56b of the receiving portion 56D. An insulating member 56f can be provided on the inner surface 56s of the receiving portion 56D in the middle part in the longitudinal axis direction A.

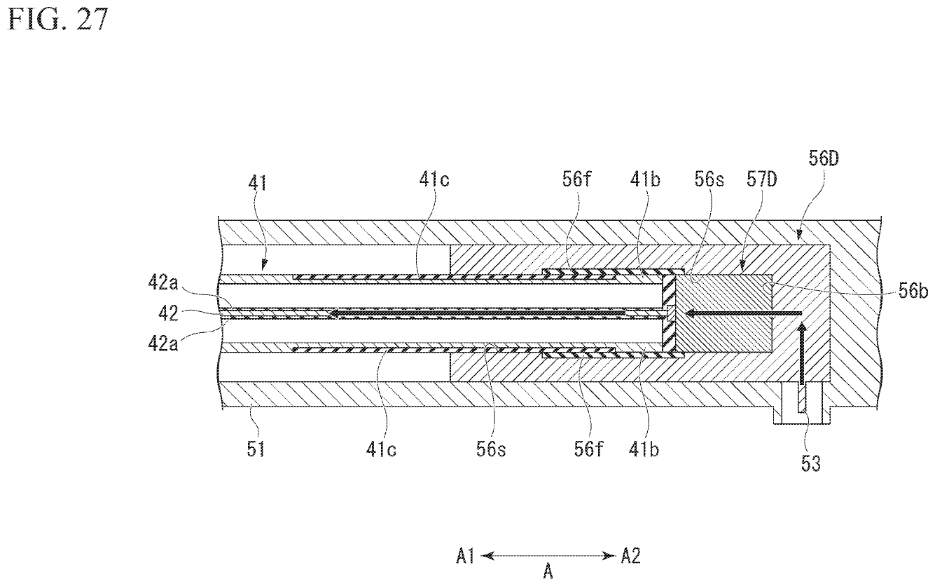

As shown in FIG. 27, the slider 52 is moved to the proximal end side A2, and the movable portion 57D is accommodated in the proximal end side A2 of the receiving portion 56D, whereby the movable portion 57D can engage with the inner surface 56s and the bottom surface 56b. The high-frequency current supplied from the power supply connector 53 can be supplied to the second rod 23 via the receiving portion 56D, the movable portion 57D, and the electric wire 42.

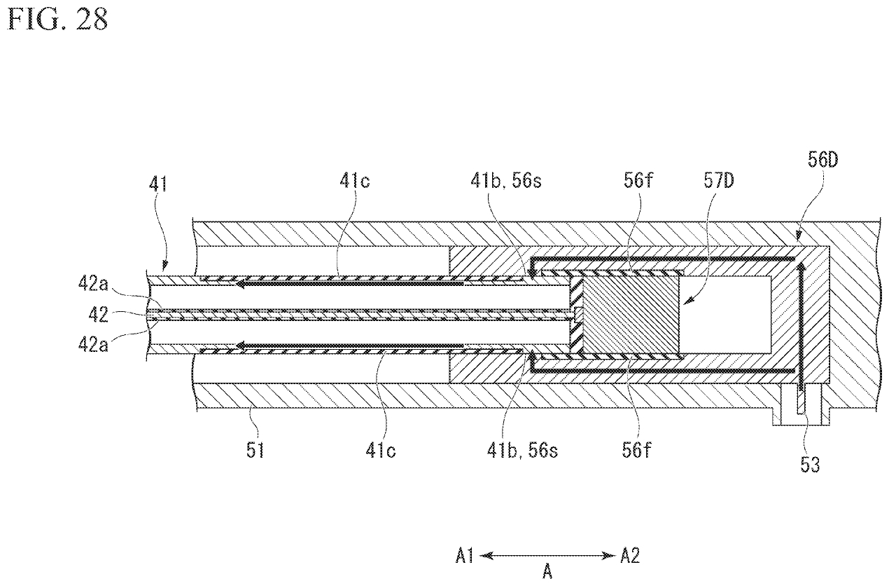

As shown in FIG. 28, by moving the slider 52 to the distal end side A1 and moving the movable portion 57D to the distal end side A1 of the receiving portion 56D, the movable portion 57D can engage with the inner surface 56s and the most proximal end 41b. High-frequency current supplied from the power supply connector 53 can be supplied to the first rod 20 and the first electrode 21 via the inner surface 56s, the most proximal end 41b, and the operating wire 41.

The receiving portion 56D and the movable portion 57D can ensure a long contactable portion in the longitudinal axis direction A, so that the electrical contact between the receiving portion 56D and the movable portion 57D is stabilized even if the length of the sheath 1 changes due to bending of the sheath 1.

### Modification 2-5

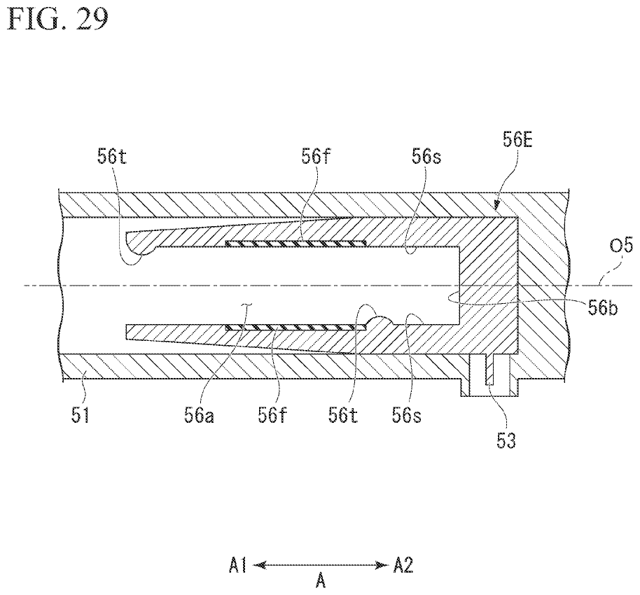

FIG. 29 shows an example of a receiving portion 56E, which is a modification of the receiving portion 56D. The receiving portion 56E can include two protrusions 56t that project inward in the radial direction R on the inner surface 56s where the insulating member 56c is not provided. One protrusion 56t can be disposed on the distal end side A1 of the insulating member 56f. The other protrusion 56t can be disposed on the proximal end side A2 of the insulating member 56f. In a front view seen from the direction along the longitudinal axis A, the two protrusions 56t can be disposed on both sides of the central axis O5 of the recess 56a. As with the receiving portion 56B, the electrical contact between the most proximal end 41b and the inner surface 56s can be stabilized. Furthermore, since the protrusion 56t on the proximal end side A2 contacts the movable portion 57D, the electrical contact between the movable portion 57D and the inner surface 56s can also be stabilized. In an example, the protrusions 56t may be ball plungers.

The present disclosure can be applied to endoscope treatment tools.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

What is claimed is:

1. An endoscope treatment tool, comprising:
a sheath;
a first rod protruding from a distal end of the sheath;
an insulator fixed to a distal end of the first rod; and
a second rod extending to a distal side from the insulator, wherein the first rod and the second rod are conductive, and wherein the second rod is fixed relative to the insulator in a position along a longitudinal axis of the first rod.

2. The endoscope treatment tool according to claim 1, wherein an outer diameter of at least a portion of the second rod is equal to or smaller than an outer diameter of the first rod.

3. The endoscope treatment tool according to claim 1, wherein a length between a distal end of the insulator and a distal end of the second rod is smaller than a protruding length of the first rod from the sheath.

4. The endoscope treatment tool according to claim 1, wherein the second rod is fixed to the insulator.

5. The endoscope treatment tool according to claim 1, wherein the second rod is connected to a wire passing through an inside portion of the first rod, and wherein the wire is conductive.

6. The endoscope treatment tool according to claim 1, further comprising:
a switch selecting one of the first rod or the second rod as a current path.

7. The endoscope treatment tool according to claim 6, further comprising:
a handle located at a proximal end of the sheath, wherein the switch located on the handle.

8. The endoscope treatment tool according to claim 6, wherein the insulator, the first rod, and the second rod are configured to integrally advance and retract as an integral part relative to the sheath, and wherein the switch selects one of the first rod or the second rod as the current path by advancing and retracting movement of the first rod.

9. The endoscope treatment tool according to claim 6, wherein the insulator, the first rod, and the second rod are configured to advance and retract as an integral part relative to the sheath, and wherein the switch selects one of the first rod or the second rod as the current path independent of advancing and retracting movement of the first rod.

10. The endoscope treatment tool according to claim 1, wherein a proximal end side of the first rod protruding from the sheath is covered with an insulating member.

11. The endoscope treatment tool according to claim 1, further comprising:
an electrode connected to a distal end of the first rod, wherein the insulator is located distally relative to electrode, and wherein at least a part of the electrode protrudes outward from an outer surface of a proximal end of the insulator in a radial direction of the first rod.

12. The endoscope treatment tool according to claim 11, wherein the electrode has a protrusion protruding outward from the outer surface of the proximal end of the insulator in the radial direction of the first rod, and wherein a maximum distance between a central axis of the second rod and an outer surface of the second rod in a front view from a longitudinal direction is smaller than a distance between a central axis of the first rod and a top of the protrusion of the electrode.

13. An endoscope treatment tool, comprising:
a sheath;
a first rod protruding from a distal end of the sheath;
an insulator located at a distal end of the first rod; and
a second rod extending from the insulator, wherein the first rod and the second rod are conductive, and wherein the insulator, the first rod, and the second rod are configured to integrally advance and retract relative to the sheath between a first position where the first rod is advanced to its fullest extent and a second position where the first rod is retracted to its fullest extent.

14. The endoscope treatment tool according to claim 13, wherein a length between a distal end of the insulator and a distal end of the second rod is smaller than a maximum amount of protrusion of the first rod from the sheath.

15. An endoscope treatment tool, comprising:
a sheath;
a first rod protruding from a distal end of the sheath;
an insulator located at a distal end of the first rod;
a second rod extending from the insulator; and
a switch configured to select one of the first rod or the second rod as an electric current path, and wherein the first rod and the second rod are conductive.

16. The endoscope treatment tool according to claim 15, wherein the second rod is connected to a wire passing through an inside portion of the first rod, and wherein the wire is conductive.

17. The endoscope treatment tool according to claim 15, further comprising:
a handle located on a proximal end of the sheath, and wherein the switch is located on the handle.

18. The endoscope treatment tool according to claim 15, wherein the switch is configured to select one of the first rod or the second rod as the electric current path by advancing and retracting of the first rod.

19. The endoscope treatment tool of claim 15, wherein the insulator, the first rod, and the second rod are integrally configured to advance and retract relative to the sheath.

20. The endoscope treatment tool of claim 15, wherein the second rod is configured to advance and retract relative to the insulator and the first rod.
